(19) United States
(12) Patent Application Publication
(10) Pub. No.: US 2023/0135680 A1
(43) Pub. Date: May 4, 2023

(54) PORTABLE HEART MONITOR SYSTEM

(71) Applicant: Eduardo Rueda, Oxnard, CA (US)

(72) Inventor: Eduardo Rueda, Oxnard, CA (US)

(21) Appl. No.: 17/514,341

(22) Filed: Oct. 29, 2021

(51) Int. Cl. A61B 5/024; A61B 5/0205; A61B 5/1455; A61B 5/0245; A61B 5/318; A61B 5/00

(52) U.S. Cl. CPC A61B 5/02438; A61B 5/0205; A61B 5/14552; A61B 5/0245; A61B 5/318; A61B 5/749; A61B 2562/222; A61B 2562/227

(57) ABSTRACT

A portable heart monitor system, including a wearable electrocardiogram device to be worn by a user to detect at least one of a heart rate, a heart rhythm, and a level of oxygen saturation, and a wearable monitoring device to be worn by the user and connected to the wearable electrocardiogram device to display at least one of the heart rate, the heart rhythm, and the level of oxygen saturation.

### BACKGROUND

### 1. Field

The present general inventive concept relates generally to heart health, and particularly, to a portable heart monitor system.

### 2. Description of the Related Art

Heart rate monitors are one of the most commonly used and beneficial pieces of equipment in the medical industry. However, the heart rate monitor can be tedious to set up due to numerous wires, and bulky monitors to display information on a heart being monitored.

Moreover, the heart rate monitor often hampers the patient's movement, thus making it difficult and uncomfortable for patients to move without feeling restricted. Also, the current design is a time-consuming process for healthcare providers during preparation of the patient for monitoring.

Therefore, there is a need for a portable heart monitoring system that simplifies set up of the heart monitor on a patient and facilitates movement while wearing the heart monitor.

### SUMMARY

The present general inventive concept provides a portable heart monitor system.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a portable heart monitor system, including a wearable electrocardiogram device to be worn by a user to detect at least one of a heart rate, a heart rhythm, and a level of oxygen saturation, and a wearable monitoring device to be worn by the user and connected to the wearable electrocardiogram device to display at least one of the heart rate, the heart rhythm, and the level of oxygen saturation.

The wearable electrocardiogram device may include an electrocardiogram body, a plurality of leads detachably connected to at least a portion of the electrocardiogram body to detect at least one of a heart rate and a heart rhythm, and a pulse oximeter disposed on at least a portion of the electrocardiogram body to detect the level of oxygen saturation.

The wearable electrocardiogram device may further include a lead connector disposed on and within at least a portion of the electrocardiogram body to magnetically receive each of the plurality of leads thereon.

The wearable electrocardiogram device may further include a lead connector disposed on and within at least a portion of the electrocardiogram body to wirelessly connect the plurality of leads to the electrocardiogram body.

The wearable monitoring device may receive at least one oral command to adjust at least one setting thereof.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a plan view of a portable heart monitor system, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

List of Components

Portable Heart Monitor System 100

Wearable Electrocardiogram Device 110

ECG Body 111

Leads 112

Lead Connector 113

Communication Unit 114

Pulse Oximeter 115

Power Button 116

Power Source 117

Wearable Monitoring Device 120

Monitor Body 121

Display Unit 122

Communication Unit 123

Power Source 124

Strap 125

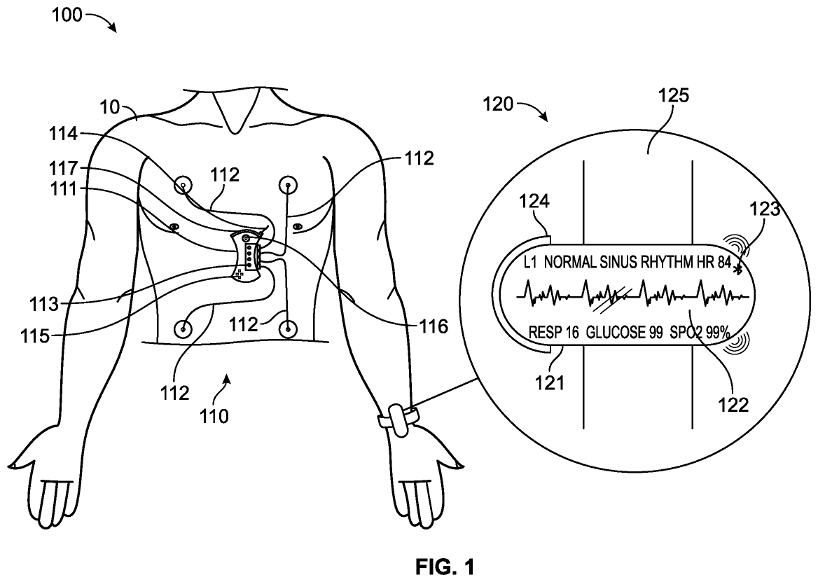

FIG. 1 illustrates a plan view of a portable heart monitor system 100, according to an exemplary embodiment of the present general inventive concept.

The portable heart monitor system 100 may be constructed from at least one of metal, plastic, wood, glass, and rubber, etc., but is not limited thereto.

The portable heart monitor system 100 may include a wearable electrocardiogram (ECG) device 110 and a wearable monitoring device 120, but is not limited thereto.

The wearable ECG device 110 may include an ECG body 111, a plurality of leads 112, a lead connector 113, a communication unit 114, a pulse oximeter 115, a power button 116, and a power source 117, but is not limited thereto.

The ECG body 111 may be removably connected to at least a portion of a body of a user 10. More specifically, the ECG body 111 may use a fastener, such as an adhesive (e.g., tape, glue). As such, the ECG body 111 may be worn by the user 10.

Referring to FIG. 1, the plurality of leads 112 is illustrated to have four leads. However, the plurality of leads 112 may have twelve leads, six leads, and/or two leads. Moreover, a number of the plurality of leads 112 may be customized based on a preference of the user 10. In other words, the plurality of leads 112 may be detachably connected to at least a portion of the ECG body 111.

Each of the plurality of leads 112 may include an electrode, but is not limited thereto. Furthermore, the plurality of leads 112 may have a type, such as limb leads, augmented limb leads, and/or precordial leads. Each of the plurality of leads 112 may be removably connected via the electrode to at least a portion of the body of the user 10 based on the type of lead corresponding to a position of the body of the user 10. Furthermore, each of the plurality of leads 112 may measure an electrical potential difference between a corresponding location of attachment of each of the plurality of leads 112. As such, the plurality of leads 112 may detect a heart rate, a heart rhythm, and/or a glucose level.

The lead connector 113 may include a plurality of jacks, but is not limited thereto.

The lead connector 113 may be disposed on and/or within at least a portion of the ECG body 111. The lead connector 113 may magnetically receive each of the plurality of leads 112 on at least one of the plurality of jacks, such that the plurality of leads 112 may magnetically connect to the ECG body 111. Alternatively, the plurality of leads 112 may be wirelessly connected to the lead connector 113.

The communication unit 114 may include a device capable of wireless or wired communication between other wireless or wired devices via at least one of Wi-Fi, Wi-Fi Direct, infrared (IR) wireless communication, satellite communication, broadcast radio communication, Microwave radio communication, Bluetooth, Bluetooth Low Energy (BLE), Zigbee, near field communication (NFC), and radio frequency (RF) communication, USB, global positioning system (GPS), Firewire, and Ethernet.

The communication unit 114 may be disposed on and/or within at least a portion of the ECG body 111. The communication unit 114 may transmit at least one lead signal therefrom. For example, the communication unit 114 may transmit data to a cardiologist to provide real time info on heart activity and facilitate deciphering cardiac emergencies.

The pulse oximeter 115 may be disposed on at least a portion of the ECG body 111. Alternatively, the pulse oximeter 115 may be a wearable device that is separate from the ECG body 111. As such, the pulse oximeter 115 may be a bracelet, an anklet, an attachment on a finger, an attachment on a foot, an attachment on an earlobe, a strap, a watch, and an armband, etc., but is not limited thereto.

Moreover, the pulse oximeter 115 may activate in response to the pulse oximeter 115 being connected to the user 10. The pulse oximeter 115 may monitor a level of oxygen saturation in blood of the user 10.

The pulse oximeter 115 may measure the level of oxygen saturation in the blood of the user 10 to detect fluctuations in the level of oxygen saturation as a result of the user 10 having difficulty breathing. Thus, the pulse oximeter 115 detecting a low level of oxygen saturation may indicate the user has difficulty breathing. Furthermore, the communication unit 114 may transmit at least one pulse oximeter signal therefrom.

The power button 116 may be disposed on at least a portion of the ECG body 111. The power button 116 may turn on the plurality of leads 112, the lead connector 113, the communication unit 114, and/or the pulse oximeter 115 in response to depressing the power button a first time. The power button 116 may turn off the plurality of leads 112, the lead connector 113, the communication unit 114, and/or the pulse oximeter 115 in response to depressing the power button a second time.

The power source 117 may include a battery and a solar cell, but is not limited thereto.

The power source 117 may be disposed within at least a portion of the ECG body 111. The power source 117 may provide power to the plurality of leads 112, the lead connector 113, the communication unit 114, the pulse oximeter 115, and/or the power button 116. Additionally, the power source 117 may be magnetically charged using induction.

The wearable monitoring device 120 may include a monitor body 121, a display unit 122, a communication unit 123, a power source 124, and a strap 125, but is not limited thereto.

Referring again to FIG. 1, the monitor body 121 is illustrated to have an oval shape. However, the monitor body 121 may be rectangular, circular, conical, triangular, pentagonal, hexagonal, heptagonal, octagonal, or any other shape known to one of ordinary skill in the art, but is not limited thereto. The monitor body 121 may be removably connected to at least a portion of the body of the user 10. In other words, the monitor body 121 may be worn by the user 10.

The display unit 122 may include a plasma screen, an LCD screen, a light emitting diode (LED) screen, an organic LED (OLED) screen, a computer monitor, a hologram output unit, a sound outputting unit, or any other type of device that visually or aurally displays data. Also, the display unit 122 may be a touch-screen that receives an input thereon.

The display unit 122 may be disposed on at least a portion of the monitor body 121. Referring again to FIG. 1, the display unit 122 may span an entire size of a side of the monitor body 121 to facilitate viewing thereof. The display unit 122 may display the heart rate, the heart rhythm, the glucose level, and/or the level of oxygen saturation. As such, the display unit 122 may display data detected by the plurality of leads 112 and/or the pulse oximeter 115.

Furthermore, the display unit 122 may receive at least one oral command to adjust at least one setting of the display unit 122. Also, the display unit 122 may receive the at least one oral command to notify a third party (e.g., a doctor, a cardiologist, a family member, a friend, etc.) during an emergency event (e.g., a heart attack, arrythmia).

The communication unit 123 may include a device capable of wireless or wired communication between other wireless or wired devices via at least one of Wi-Fi, Wi-Fi Direct, infrared (IR) wireless communication, satellite communication, broadcast radio communication, Microwave radio communication, Bluetooth, Bluetooth Low Energy (BLE), Zigbee, near field communication (NFC), and radio frequency (RF) communication, USB, global positioning system (GPS), Firewire, and Ethernet.

The communication unit 123 may be disposed within at least a portion of the monitor body 121. The communication unit 123 may wirelessly receive the at least one lead signal and/or the at least one pulse oximeter signal from the communication unit 114. Accordingly, the display unit 122 may display data detected from the plurality of leads 112 and/or the pulse oximeter 115, and received from the communication unit 123.

The power source 124 may include a battery and a solar cell, but is not limited thereto.

The power source 124 may be disposed within at least a portion of the ECG body 111. The power source 124 may provide power to the display unit 122 and/or the communication unit 123. Additionally, the power source 124 may be magnetically charged using induction.

The strap 125 may be disposed on at least a portion of the monitor body 121. Therefore, the strap 125 may facilitate wearing of the monitor body 121 by the user 10.

Therefore, the portable heart monitor system 100 may facilitate movement by the user 10 due to having less wires. Also, the portable heart monitor system 100 requires less management by health care workers to connect the portable heart monitor system 100 to the user 10.

The present general inventive concept may include a portable heart monitor system 100, including a wearable electrocardiogram device 110 to be worn by a user to detect at least one of a heart rate, a heart rhythm, and a level of oxygen saturation, and a wearable monitoring device 120 to be worn by the user and connected to the wearable electrocardiogram device 110 to display at least one of the heart rate, the heart rhythm, and the level of oxygen saturation.

The wearable electrocardiogram device 110 may include an electrocardiogram body 111, a plurality of leads 112 detachably connected to at least a portion of the electrocardiogram body 111 to detect at least one of a heart rate and a heart rhythm, and a pulse oximeter 115 disposed on at least a portion of the electrocardiogram body 111 to detect the level of oxygen saturation.

The wearable electrocardiogram device 110 may further include a lead connector 113 disposed on and within at least a portion of the electrocardiogram body 111 to magnetically receive each of the plurality of leads 112 thereon.

The wearable electrocardiogram device 110 may further include a lead connector 113 disposed on and within at least a portion of the electrocardiogram body 111 to wirelessly connect the plurality of leads 112 to the electrocardiogram body 111.

The wearable monitoring device 120 may receive at least one oral command to adjust at least one setting thereof.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A portable heart monitor system, comprising:
a wearable electrocardiogram device to be worn by a user to detect at least one of a heart rate, a heart rhythm, and a level of oxygen saturation; and
a wearable monitoring device to be worn by the user and connected to the wearable electrocardiogram device to display at least one of the heart rate, the heart rhythm, and the level of oxygen saturation.

2. The portable heart monitor system of claim 1, wherein the wearable electrocardiogram device comprises:
an electrocardiogram body;
a plurality of leads detachably connected to at least a portion of the electrocardiogram body to detect at least one of a heart rate and a heart rhythm; and
a pulse oximeter disposed on at least a portion of the electrocardiogram body to detect the level of oxygen saturation.

3. The portable heart monitor system of claim 2, wherein the wearable electrocardiogram device further comprises:
a lead connector disposed on and within at least a portion of the electrocardiogram body to magnetically receive each of the plurality of leads thereon.

4. The portable heart monitor system of claim 2, wherein the wearable electrocardiogram device further comprises:
a lead connector disposed on and within at least a portion of the electrocardiogram body to wirelessly connect the plurality of leads to the electrocardiogram body.

5. The portable heart monitor system of claim 1, wherein the wearable monitoring device receives at least one oral command to adjust at least one setting thereof.
